(19) United States
(12) Patent Application Publication
(10) Pub. No.: US 2006/0074396 A1
(43) Pub. Date: Apr. 6, 2006

(54) Stent delivery system

(75) Inventor: Mark L. Stiger, Windsor, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 10/952,630

(22) Filed: Sep. 28, 2004

(51) Int. Cl. A61M 31/00

(52) U.S. Cl. 604/509; 427/2.1; 623/1.11; 264/563

(57) ABSTRACT

The invention provides a system for treating a vascular condition, a method of manufacturing the system, and a method for using the system to deliver a therapeutic agent to a desired location in a bodily vessel. The system comprises a tubular inner member received in a lumen of an elongated catheter shaft and extending beyond a distal end of the catheter shaft. The system includes at least one inflatable balloon positioned between distal and proximal perfusion ports. At least a portion of the balloon is disposed on the inner member, at least a portion of the inner member underlying the balloon having a wall thickness between about 0.0015 inch and 0.0025 inch and an inner diameter between about 0.020 inch and 0.040 inch. A stent is removably coupled to the balloon. A therapeutic agent is disposed on at least a portion of the stent.

### TECHNICAL FIELD

This invention relates generally to treatment of vascular conditions. More specifically, the invention relates to a high-flow perfusion catheter and methods for making and using the same.

### BACKGROUND OF THE INVENTION

Heart disease, specifically coronary artery disease, is a major cause of death, disability, and healthcare expense in the United States and other industrialized countries. A number of methods and devices for treating coronary heart disease have been developed, some of which are specifically designed to treat the complications resulting from atherosclerosis and other forms of coronary arterial narrowing.

One method for treating such conditions is percutaneous transluminal coronary angioplasty (PTCA). During PTCA, a balloon catheter device is inflated to dilate a stenotic blood vessel. The stenosis may be the result of a lesion such as a plaque or thrombus. When inflated, the pressurized balloon exerts a compressive force on the lesion, thereby increasing the inner diameter of the affected vessel. The increased interior vessel diameter facilitates improved blood flow. A disadvantage of many balloon catheter devices is that during inflation, the balloon occludes the vessel, cutting off the flow of blood and limiting the amount of time the balloon may remain inflated without resultant damage to the vessel and adjacent tissue.

Soon after the procedure, a significant proportion of treated vessels restenose. To prevent restenosis, a stent, constructed of a metal or polymer, is implanted within the vessel to maintain lumen size. The stent acts as a scaffold to support the lumen in an open position. Configurations of stents include a cylindrical tube defined by a mesh, interconnected stents, or like segments. Exemplary stents are disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau.

Stent insertion may cause undesirable reactions such as inflammation, infection, thrombosis, and proliferation of cell growth that occludes the passageway. Therapeutic agents that assist in preventing these conditions have been delivered to the site by coating these agents onto a stent. Current stent delivery methods allow a substantial percentage of the drug coating on both the inner and outer surfaces of the stent to be washed away and lost into the blood stream during and after delivery of the stent to a treatment site.

Finally, it is within reason to assume that future therapies may include devices that dictate the use of a catheter or delivery system that can be inflated and allowed to dwell in a body lumen for an extended period of time.

Therefore, it would be desirable to have a high-flow perfusion catheter and methods for making and using such a catheter that overcome the aforementioned and other disadvantages.

### SUMMARY OF THE INVENTION

One aspect of the present invention is a system for treating a vascular condition, comprising an elongated catheter shaft, a tubular inner member, and an inflatable balloon. The inner member is received in a lumen of the catheter shaft and extends beyond a distal end of the catheter shaft. A distal portion of the catheter shaft includes at least one perfusion opening. The inner member includes at least one perfusion opening in communication with the at least one catheter shaft perfusion opening to form at least one proximal perfusion port. The inner member also includes at least one distal perfusion port. The proximal and distal perfusion ports are in communication with an inner member lumen. At least a portion of the inflatable balloon is disposed on the inner member. At least a portion of the inner member underlying the balloon has a wall thickness between about 0.0015 inch and 0.0025 inch and an inner diameter between about 0.020 inch and 0.040 inch.

Another aspect of the present invention is a method for delivering a therapeutic agent to a desired location in a bodily vessel. A perfusion catheter is provided. The catheter comprises a tubular inner member received in a lumen of an elongated catheter shaft and extending beyond a distal end of the catheter shaft. The catheter includes distal and proximal perfusion ports in communication with an inner member lumen. The catheter includes an inflatable balloon, at least a portion of the balloon disposed on the inner member. At least a portion of the inner member underlying the balloon has a wall thickness between about 0.0015 inch and 0.0025 inch and an inner diameter between about 0.020 inch and 0.040 inch. The catheter includes a stent removably coupled to the balloon. The stent includes a therapeutic agent disposed on at least a portion of the stent. The catheter is delivered to the desired location in the vessel. The balloon is inflated such that the stent contacts the wall of the vessel, the balloon sealing the stent against the wall of the vessel. A fluid contained in the vessel flows through the perfusion ports and through the inner member to provide perfusion of the vessel proximal and distal to the balloon. Inflation of the balloon is maintained for a period of at least 15 seconds. The balloon is deflated, leaving the stent deployed within the vessel. The catheter is removed from the vessel.

Yet another aspect of the present invention is a method of manufacturing a system for treating a vascular condition. An elongated catheter shaft and a tubular inner member are provided. The inner member is placed within a lumen of the catheter shaft such that the inner member extends beyond a distal end of the catheter shaft. A distal portion of the catheter shaft is bonded to an adjacent portion of the inner member to form a bonded portion. At least one perfusion opening is formed in the catheter shaft, and at least one perfusion opening is formed in the inner member. The at least one inner member perfusion opening is positioned in communication with the at least one catheter shaft perfusion opening to form at least one proximal perfusion port. At least one distal perfusion port is formed in a distal portion of the inner member. A balloon is provided. A proximal end of the balloon is coupled to the catheter shaft. A distal end of the balloon is coupled to the inner member.

The aforementioned and other features and advantages of the invention will become further apparent from the following detailed description of the presently preferred embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE DRAWINGS

FIG. 1 is a longitudinal, partially cross-sectional view of one embodiment of a system for treating a vascular condition, in accordance with the present invention;

FIG. 2 is a transverse cross-sectional view of one portion of the system of FIG. 1;

FIG. 3 is a flow diagram of one embodiment of a method for delivering a therapeutic agent to a desired location in a bodily vessel, in accordance with the present invention; and

FIG. 4 is a flow diagram of one embodiment of a method of manufacturing a system for treating a vascular condition, in accordance with the present invention.

### DETAILED DESCRIPTION OF THE PRESENTLY PREFERRED EMBODIMENTS

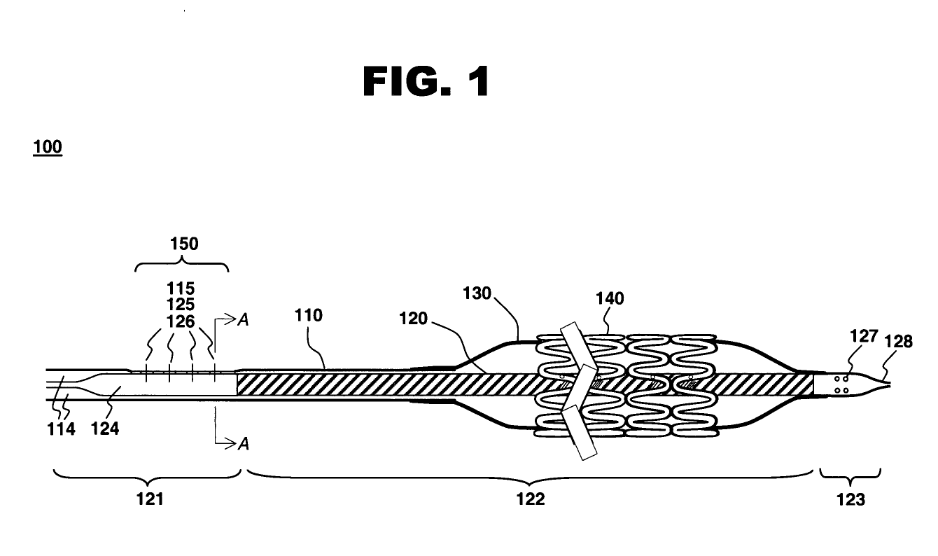

One aspect of the present invention is a system for treating a vascular condition. FIG. 1 shows one embodiment of the system at 100, in accordance with the present invention. System 100 comprises an elongated catheter shaft 110, a tubular inner member 120, an inflatable balloon 130, and a stent 140. The system may also include a sheath (not shown) that removably covers the stent to provide restraint and/or protection for the stent.

Elongated catheter shaft 110 may be made using one or more suitable materials known in the art. In the current embodiment, a proximal segment of the catheter shaft is made from a polymer-coated stainless steel hypotube to provide controllability and push force transmission to the distal segment of the catheter shaft. The distal segment is made from a material having greater flexibility, such as a single- or multi-layer polymer tubing. To enhance lubricity of the system, a hydrophilic coating may be disposed on at least a portion of the exterior surface of catheter shaft 110.

FIG. 1 shows only a distal portion of system 100. It is within this portion that perfusion occurs. As shown, tubular inner member 120 comprises a proximal segment 121, a composite segment 122, and a distal tip segment 123 that includes distal tip 128. The proximal 121 and composite 122 segments of inner member 120 are formed from different materials, with proximal segment 121 comprising a highly flexible tri-layer polymer tubing, while composite segment 122 comprises a somewhat stiffer but less compressible wire mesh and polymer composite that has a wall thickness of about 0.002 inch and an inner diameter of about 0.035 inch. Alternatively, composite segment 122 may comprise a spiral round ribbon or wire within a polymer material. Two or more ribbons or wires may be used for a braided configuration.

As the present embodiment is shown in FIG. 1, the inner and outer diameters of proximal segment 121 are larger at the distal end portion of the segment, where proximal segment 121 joins composite segment 122, than over the elongated proximal portion of the segment. The increased inner diameter, in particular, improves perfusion, while the narrower profile of most of proximal segment 121 offers optimum flexibility. Distal tip segment 123 is formed using the same tri-layer polymer tubing used to form proximal segment 121. One skilled in the art will recognize that alternative embodiments may comprise other materials. For example, the material used to form distal tip segment 123 may be chosen to provide desired crossing characteristics, i.e., a hard material for distal tip 128 if the catheter tip must cross through a hardened lesion within a vessel, a soft material if flexibility is the primary requirement.

Proximal segment 121 is received in a lumen 114 of catheter shaft 110. In the present embodiment, a portion of composite segment 122 and all of distal tip segment 123 extend beyond the distal end of catheter shaft 110, while proximal segment 121 is fully within the lumen of catheter shaft 110. Catheter shaft 110 includes perfusion openings 115, formed in a distal portion of the shaft. Inner member proximal segment 121 includes perfusion openings 125, which are in communication with the catheter shaft perfusion openings. Together, the inner member and catheter shaft perfusion openings form proximal perfusion ports 126.

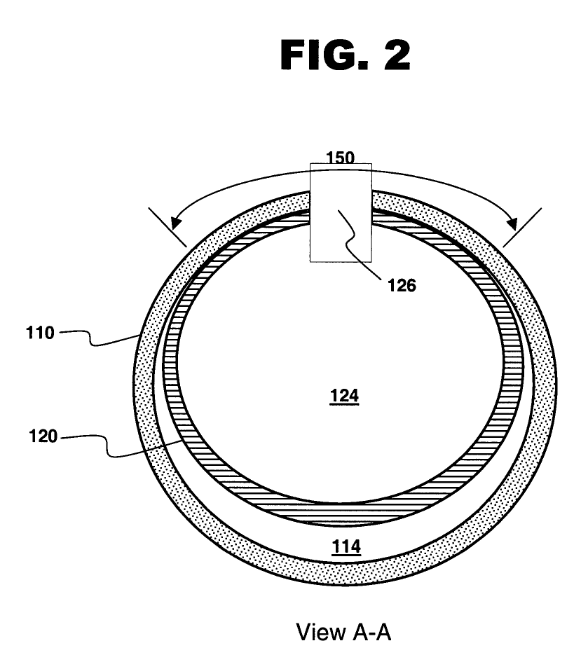

Catheter shaft 110 is shown bonded to inner member 120 in the area of the perfusion openings, forming bonded portion 150. As seen in FIG. 2, in which like elements share like numbers with FIG. 1, bonded portion 150 occludes only a portion of the annular space between catheter shaft 110 and inner member 120. The size of bonded portion 150 may be varied depending on the number and size of proximal perfusion ports desired.

Distal perfusion ports 127 are formed in distal tip segment 123. The proximal and distal perfusion ports are in communication with lumen 124 of inner member 120, permitting a fluid such as blood, cerebrospinal fluid, or urea to flow in through the ports on one side of composite segment 122, through the composite segment, and out through the ports on the opposite side of the composite segment. In the present embodiment, composite segment 122 has a relatively large inner diameter of about 0.035 inch, which permits fluid with a specific gravity of 1.110 to flow at a rate of about forty cubic centimeters per minute (40 cc/min) through the system. Inner member lumen 124 may also serve as a guidewire lumen during delivery of the system to a treatment site. The guidewire may be withdrawn proximally to perfusion ports 126 to facilitate flow of a fluid through the perfusion portion of system 100, i.e., that portion including the distal end portion of segment 121 and segments 122, and 123.

In the present embodiment, each catheter shaft perfusion opening 115 is formed at essentially the same time as the corresponding inner member perfusion opening 125 after catheter shaft 110 has been bonded to inner member 120. This may be accomplished by, for example, drilling the openings through the bonded catheter shaft 110 and inner member 120. The resulting proximal perfusion ports 126, four in the present embodiment, each about 0.015 inch to 0.016 inch in diameter, are arranged in a linear configuration with a hole spacing of about 0.5 millimeters. Six distal perfusion ports 127 are arranged in three groups of two around the circumference of distal tip segment 123. The distal perfusion ports are also about 0.015 inch to 0.016 inch in diameter.

As will be apparent to one skilled in the art, the number, size, shape, and orientation of the perfusion openings and perfusion ports may be varied as needed. For example, in an alternative embodiment, a single catheter shaft perfusion opening may communicate with multiple inner member perfusion openings to form multiple proximal perfusion ports, and any or all of the perfusion openings and perfusion ports may be vertical slots, horizontal slits, and the like.

Inflatable balloon 130 is coupled to both the catheter shaft and the inner member, with the proximal end of balloon 130 coupled to catheter shaft 110 distal to proximal perfusion ports 126, and the distal end of balloon 130 coupled to inner member 120 proximal to distal perfusion ports 127. The body of balloon 130 overlies inner member segment 122, which, as noted above, comprises a composite of wire mesh and polymer and is, therefore, capable of withstanding the inflation pressures acting on the outer surface of the segment during inflation of balloon 130. As can be seen in FIG. 1, lumen 114 of catheter shaft 110 opens into balloon 130 and serves as an inflation lumen for the balloon.

Inflatable balloon 130 may be any balloon known in the art that is appropriate for delivering a stent to a treatment site, for example one made of a material such as polyethylene, polyethylene terephthalate (PET), nylon, nylon co-polymer, or the like.

Stent 140 may comprise a variety of medical implantable materials, such as stainless steel, nitinol, tantalum, ceramic, nickel, titanium, aluminum, polymeric materials, MP35N, stainless steel, titanium ASTM F63-83 Grade 1, niobium, high carat gold K 19-22, or combinations of the above. Stent 140 includes a therapeutic agent disposed on the stent. The therapeutic agent may be, for example, an antiproliferative agent, an antineoplastic agent, an antibiotic agent, an anti-inflammatory agent, a free radical scavenger, a protein, combinations thereof, and the like.

Stent 140 is removably coupled to balloon 130, with the balloon extending beyond both the proximal and distal ends of the stent. When balloon 130 is inflated, stent 140 expands and the areas of balloon 130 that extend beyond stent 140 seal against the wall of the vessel on either side of the stent, trapping the area of treatment. Thus, a therapeutic agent disposed on stent 140 may be absorbed by the wall of the vessel undisturbed by the fluid flowing through the vessel. Because system 100 allows the vessel to be adequately perfused both distal and proximal to balloon 130, the balloon may remain inflated as long as is necessary to transfer the desired amount of therapeutic agent from stent 140 to the vessel wall.

System 100 may include a sheath (not shown). The sheath is positioned over the stent during delivery of the stent to a treatment location and is removed, for example retracted, for deployment of the stent. The sheath may be used with a self-expanding stent to restrain the stent and may also be used with both self-expanding and expandable stents to protect the therapeutic coating from damage or loss during delivery of the stent.

One skilled in the art will recognize that, although described above in the context of a stent delivery system, system 100 may be readily adapted to other types of balloon catheters, including those having additional functionalities, structures, or intended uses. For example, because the system permits a fluid contained in the vessel to flow at a high rate through the system and past the balloon, the system may be used without the stent to apply pressure to the walls of a vessel over an extended period of time.

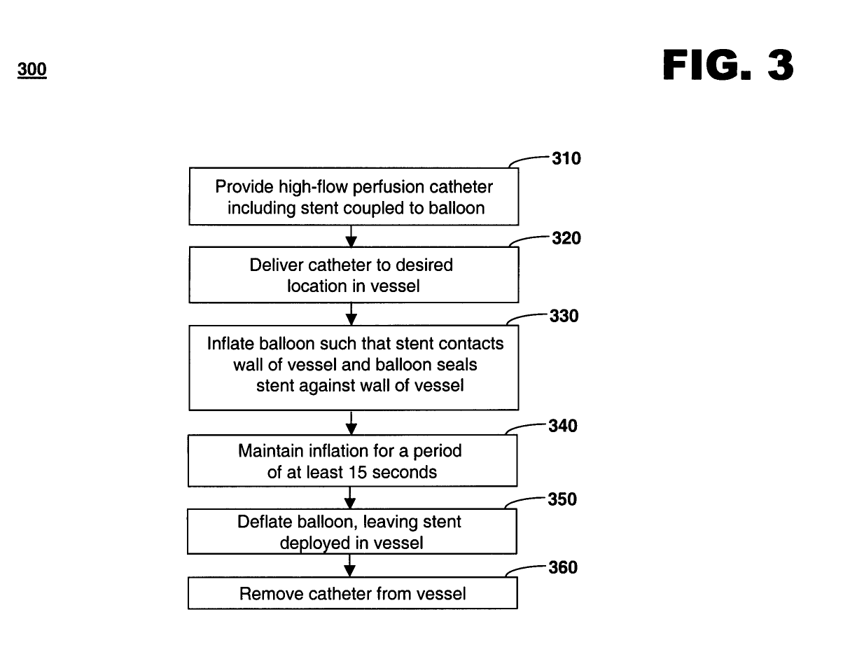

Another aspect of the present invention is a method for delivering a therapeutic agent to a desired location in a bodily vessel. FIG. 3 shows a flow diagram of one embodiment in accordance with the present invention at 300.

A high-flow perfusion catheter is provided (Block 310). The catheter comprises a tubular inner member received in a lumen of an elongated catheter shaft and extending beyond a distal end of the catheter shaft. The catheter includes distal and proximal perfusion ports, which open into a lumen of the inner member. An inflatable balloon is positioned between the distal and proximal perfusion ports with at least a portion of the balloon disposed on the inner member. At least a portion of the inner member underlying the balloon has a wall thickness between about 0.0015 inch and 0.0025 inch and an inner diameter between about 0.020 inch and 0.040 inch. A stent bearing a therapeutic agent is removably coupled to the balloon.

The catheter is delivered to the desired location in the vessel (Block 320). For example, the catheter may be introduced through a percutaneous access site and advanced over a guidewire to a position adjacent to the desired location. Once the catheter is in position, the guidewire is withdrawn to a position proximal to the catheter's proximal perfusion ports.

The balloon is inflated (Block 330) such that the stent contacts the wall of the vessel and the balloon seals the stent against the wall of the vessel, preventing flow of a fluid such as blood over the stent. Instead, the blood flows through the catheter perfusion ports and inner member lumen, providing perfusion of the vessel proximal and distal to the balloon while preventing the therapeutic agent carried by the stent from being washed away by the flow of blood past the stent.

Inflation is maintained for a period of at least 15 seconds, but typically for longer than 15 seconds (Block 340). Because the vessel is adequately perfused both distal and proximal to the balloon, the balloon may remain inflated for an extended period to transfer the desired amount of therapeutic agent from the stent to the vessel wall.

Once the therapeutic agent has been delivered, the balloon is deflated (Block 350), leaving the stent deployed within the vessel. The catheter is then removed from the vessel (Block 360).

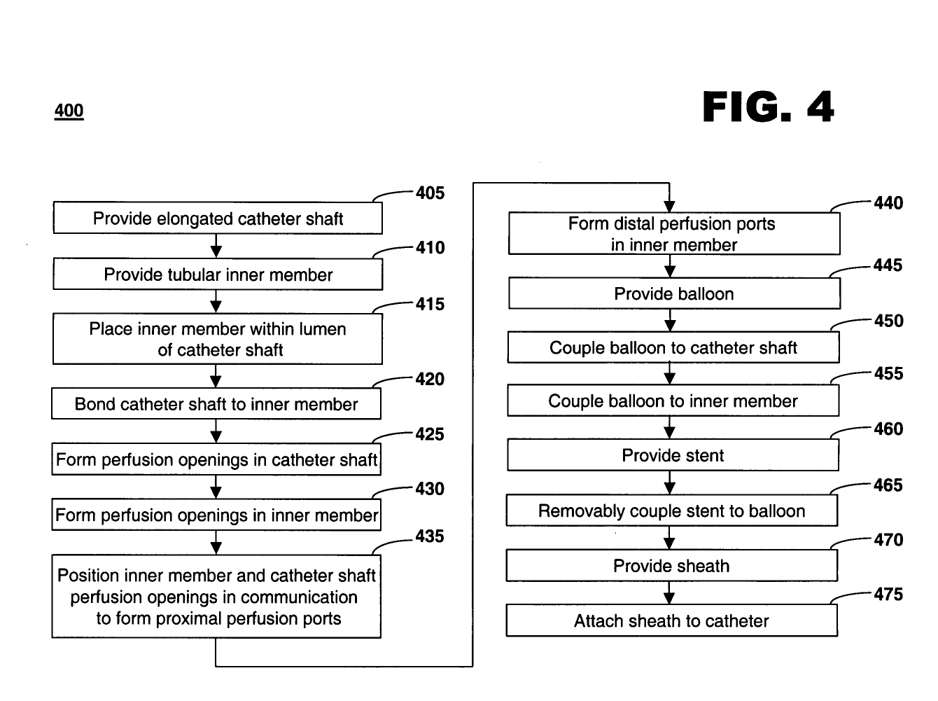

Yet another aspect of the present invention is a method of manufacturing a system for treating a vascular condition. FIG. 4 shows a flow diagram of one embodiment in accordance with the present invention at 400.

An elongated outer catheter shaft is provided (Block 405). The catheter shaft may comprise one or more suitable materials known in the art. For example, a proximal segment of the catheter shaft may be made from a polymer-coated stainless steel hypotube, while a distal segment of the catheter shaft may be made from a material having greater flexibility, such as a single- or multi-layer polymer tubing. To enhance lubricity of the system, a hydrophilic coating may be applied to at least a portion of the exterior surface of the catheter shaft.

A tubular inner member is provided (Block 410). Preferably the inner member comprises a proximal segment, a composite segment, and a distal tip segment, the composite segment comprising a material different from that comprising the proximal segment. The segments may be assembled by flaring out the distal end of one segment to overlap the proximal end of the adjoining segment, and then using a heat block or a laser, for example, to thermally bond the distal end of the proximal segment to the proximal end of the composite segment and the distal end of the composite segment to the proximal end of the distal tip segment. If the segments comprise materials that are not thermally compatible, an adhesive may be used to bond the segments.

The proximal segment may be formed using a material chosen primarily for its flexibility, for example a single- or multi-layer polymer tubing. The composite segment, a portion of which is enclosed by the inflatable balloon, should be incompressible under balloon inflation pressures and have a large central lumen that permits high flow of a bodily fluid through this segment of the inner member. An appropriate material for this purpose is a tubing that is a composite of a wire mesh and a polymer and that has a wall thickness of about 0.002 inch and an inner diameter of about 0.035 inch. The material comprising the distal tip segment may be chosen to provide the catheter with the desired crossing characteristics, for example, a hard material if the catheter tip must cross through a hardened lesion within a vessel, or a softer material if tip flexibility is the primary requirement. The distal tip segment may be shaped into a catheter tip either before or after attaching the segment to the other segments of the inner member.

The inner member is placed within a lumen of the catheter shaft such that the inner member extends beyond a distal end of the catheter shaft (Block 415). The fully assembled inner member may be inserted within the catheter shaft; or the proximal and composite segments may be bonded to each other and inserted into the catheter shaft, and the distal tip segment may be added later.

A distal portion of the catheter shaft is bonded to the adjacent inner member to form a bonded portion (Block 420). The bond may be formed by heating the portion to be bonded, resulting in a thermal bond between the inner surface of the catheter shaft and the outer surface of the inner member. The bond occludes only a portion of the catheter shaft lumen, leaving a portion open to serve as a balloon inflation lumen.

At least one perfusion opening is formed in the catheter shaft (Block 425), and at least one perfusion opening is formed in the inner member (Block 430). Preferably the catheter shaft is first bonded to the inner member and then each catheter shaft and inner member perfusion opening is formed in the bonded portion in a single step. This ensures the catheter shaft and inner member perfusion openings are positioned in communication with each other to form at least one proximal perfusion port (Block 435). The ports may be formed by, for example, drilling or laser cutting the ports through the bonded portion and into the inner member lumen. The catheter and inner member proximal perfusion ports may also be formed separately before the catheter and inner member are assembled and bonded, and positioned in communication during assembly. In either case, the catheter and inner member proximal perfusion ports reside in the bonded portion of the system. The number, size, shape, and orientation of the perfusion ports may be varied as needed. In addition, the size of the bonded portion may be varied depending on the number and size of the ports that are required to achieve the desired perfusion, and the size of the inflation lumen that is required to inflate the balloon.

At least one distal perfusion port is formed in a distal portion of the inner member, for example in the distal tip segment (Block 440). Where the port is formed in the distal tip segment, the at least one port may be formed either before or after the distal tip segment is bonded to the composite segment, using a method such as drilling or laser cutting.

A balloon is provided (Block 445). The balloon may be made of a suitable material such as polyethylene, polyethylene terephthalate (PET), or the like. A proximal end of the balloon is coupled to the catheter shaft distal to the proximal perfusion ports (Block 450), and a distal end of the balloon is coupled to the inner member proximal to the distal perfusion ports (Block 455).

A stent is provided (Block 460). The stent may be either an expandable or self-expanding stent and may include a therapeutic agent disposed on at least a portion of the stent. The stent is removably coupled to the balloon by, for example, crimping the stent onto the balloon (Block 465).

A sheath capable of removably covering the stent is provided (Block 470) and attached to the catheter (Block 475). The sheath may be attached to the catheter either before or after coupling the stent to the balloon.

One skilled in the art will appreciate that the system may be manufactured without the sheath or without both the stent and the sheath.

While the embodiments of the invention disclosed herein are presently considered to be preferred, various changes and modifications can be made without departing from the spirit and scope of the invention. The scope of the invention is indicated in the appended claims, and all changes and modifications that come within the meaning and range of equivalents are intended to be embraced therein.

What is claimed is:

1. A system for treating a vascular condition, comprising:
an elongated catheter shaft, a distal portion of the catheter shaft including at least one perfusion opening;
a tubular inner member received in a lumen of the catheter shaft and extending beyond a distal end of the catheter shaft, the inner member including at least one perfusion opening in communication with the at least one catheter shaft perfusion opening to form at least one proximal perfusion port, the inner member including at least one distal perfusion port, the proximal and distal perfusion ports in communication with an inner member lumen; and
an inflatable balloon, at least a portion of the balloon disposed on the inner member, wherein at least a portion of the inner member underlying the balloon has a wall thickness between about 0.0015 inch and 0.0025 inch and an inner diameter between about 0.020 inch and 0.040 inch.

2. The system of claim 1 wherein a portion of the catheter shaft is bonded to a portion of the inner member to form a bonded portion, the bonded portion including the at least one inner member perfusion opening in communication with the at least one catheter shaft perfusion opening.

3. The system of claim 1 wherein a proximal end of the balloon is coupled to the catheter shaft and a distal end of the balloon is coupled to the inner member.

4. The system of claim 1 wherein the inner member comprises a proximal segment, a composite segment, and a distal tip segment, the composite segment comprising a material different from that comprising the proximal segment.

5. The system of claim 4 wherein the composite segment comprises a material having a wall thickness between about 0.0015 inch and 0.0025 inch and an inner diameter between about 0.020 inch and 0.040 inch.

6. The system of claim 1 further comprising:
a stent removably coupled to the balloon.

7. The system of claim 6 wherein the stent includes a therapeutic agent disposed on at least a portion of the stent.

8. The system of claim 6 wherein the balloon extends beyond a proximal and a distal end of the stent.

9. The system of claim 6 further comprising:
a sheath removably covering the stent.

10. The system of claim 1 wherein the catheter shaft includes a hydrophilic coating disposed on at least a portion of an exterior surface of the catheter shaft.

11. A method for delivering a therapeutic agent to a desired location in a bodily vessel, comprising:
providing a perfusion catheter comprising a tubular inner member received in a lumen of an elongated catheter shaft and extending beyond a distal end of the catheter shaft, the catheter including proximal and distal perfusion ports in communication with an inner member lumen, the catheter including an inflatable balloon, at least a portion of the balloon disposed on the inner member, at least a portion of the inner member underlying the balloon having a wall thickness between about 0.0015 inch and 0.0025 inch and an inner diameter between about 0.020 inch and 0.040 inch, the catheter including a stent removably coupled to the balloon, the stent including a therapeutic agent disposed on at least a portion of the stent;
delivering the catheter to the desired location in the vessel;
inflating the balloon such that the stent contacts the wall of the vessel, the balloon sealing the stent against the wall of the vessel, a fluid contained in the vessel flowing through the perfusion ports and through the inner member to provide perfusion of the vessel proximal and distal to the balloon;
maintaining inflation of the balloon for a period of at least 15 seconds;
deflating the balloon, leaving the stent deployed within the vessel; and
removing the catheter from the vessel.

12. The method of claim 11 wherein the catheter includes a sheath removably covering the stent.

13. The method of claim 12 further comprising:
removing the sheath from the stent prior to inflating the balloon.

14. A method of manufacturing a system for treating a vascular condition, comprising:
providing an elongated catheter shaft;
providing a tubular inner member;
placing the inner member within a lumen of the catheter shaft such that the inner member extends beyond a distal end of the catheter shaft;
bonding a portion of the catheter shaft to a portion of the inner member to form a bonded portion;
forming at least one perfusion opening in the catheter shaft;
forming at least one proximal perfusion opening in the inner member;
positioning the at least one inner member perfusion opening in communication with the at least one catheter shaft perfusion opening to form at least one proximal perfusion port;
forming at least one distal perfusion port in the inner member;
providing a balloon;
coupling a proximal end of the balloon to the catheter shaft; and
coupling a distal end of the balloon to the inner member.

15. The method of claim 14 wherein the inner member includes a proximal segment, a composite segment, and a distal tip segment, the composite segment comprising a material different from that comprising the proximal segment, and wherein providing said inner member comprises:
bonding a distal end of the proximal segment to a proximal end of the composite segment; and
bonding a distal end of the composite segment to a proximal end of the distal tip segment.

16. The method of claim 14 further comprising:
shaping a distal end of the inner member into a catheter tip.

17. The method of claim 14 further comprising:
applying a hydrophilic coating to at least a portion of an exterior surface of the catheter shaft.

18. The method of claim 14 further comprising:
providing a stent; and
removably coupling the stent to the balloon.

19. The method of claim 18 wherein the stent includes a therapeutic agent disposed on at least a portion of the stent.

20. The method of claim 18 further comprising:
providing a sheath; and
attaching the sheath to the catheter, the sheath capable of removably covering the stent.
